(19) United States
(12) Patent Application Publication
(10) Pub. No.: US 2008/0097175 A1
(43) Pub. Date: Apr. 24, 2008

(54) System and method for display control of patient monitor

(76) Inventors: Robin S. Boyce, Pleasanton, CA (US); Hui Wang, San Ramon, CA (US); Scott Amundson, Oakland, CA (US); Li Li, Petaluma, CA (US); Tonia N. Madere, Stockton, CA (US); James Ochs, San Francisco, CA (US); Steve Vargas, Pleasanton, CA (US)

Correspondence Address: Covidien;IP Counsel - Respiratory & Monitoring Solutions, 60 Middletown Avenue, North Haven, CT, 06473, US

(21) Appl. No.: 11/540,379

(22) Filed: Sep. 29, 2006

(51) Int. Cl. A61B 5/00

(52) U.S. Cl. 600/323

(57) ABSTRACT

Embodiments of the present invention relate to monitoring systems. One embodiment includes a monitoring system comprising a monitor configured to receive input relating to patient physiological parameters and to store historical data related to the parameters. Further, the system comprises a screen configured to display the historical data corresponding to the patient physiological parameters, and a display control feature configured to automatically find and display an event in the historical data on the screen when the display control feature is activated.

### BACKGROUND OF THE INVENTION

1. Field of the Invention

The present invention relates generally to a user-interface application for a patient monitoring device. In particular, present embodiments relate to a display control feature that facilitates historical data observation with a patient physiological data monitoring instrument.

2. Description of the Related Art

This section is intended to introduce the reader to various aspects of art that may be related to various aspects of the present invention, which are described and/or claimed below. This discussion is believed to be helpful in providing the reader with background information to facilitate a better understanding of the various aspects of the present invention. Accordingly, it should be understood that these statements are to be read in this light, and not as admissions of prior art.

Patient monitors include medical devices that facilitate measurement and observation of patient physiological data. For example, pulse oximeters are a type of patient monitor. A typical patient monitor cooperates with a sensor to detect and display a patient's vital signs (e.g., temperature, pulse rate, respiratory rate) and/or other physiological measurements (e.g., water content of tissue, blood oxygen level) for observation by a user (e.g., clinician). For example, pulse oximeters are generally utilized with related sensors to detect and monitor a patient's functional oxygen saturation of arterial hemoglobin (i.e., SpO₂) and pulse rate. Other types of patient monitors may be utilized to detect and monitor other physiological parameters. The use of patient monitors may improve patient care by facilitating supervision of a patient without continuous attendance by a human observer (e.g., a nurse or physician).

A patient monitor may include a screen that displays information relating to operation and use of the patient monitor. A typical patient monitor screen may display operational data that is instructive and that facilitates operation of the monitor by a user. For example, the operational data may include status indicators and instructional data relating to the monitor itself and/or monitor applications (e.g., a power indicator, an alarm silenced icon, and a battery low indicator). The screen may also display measurement data from a patient being monitored. For example, the measurement data may include information relating to a physiological feature of the patient being monitored. Specifically, the screen may display a graph or trend (e.g., a pulse rate trend, and/or a plethysmographic waveform) of data relating to particular measured physiological parameters. Such trends include historical data that may span short or long periods of time in which the particular parameter (e.g., SpO₂, pulse rate) being trended was observed. This historical data can be beneficial for handling and detecting patient issues. However, analysis of this historical information can be inconvenient due to the quantity of the information. Further, such analysis can be difficult because certain aspects of the information are difficult for a user to detect.

### BRIEF DESCRIPTION OF THE DRAWINGS

Advantages of the invention may become apparent upon reading the following detailed description and upon reference to the drawings in which:

FIG. 1 is a perspective view of a patient monitor in accordance with an exemplary embodiment of the present invention;

FIG. 2 is a perspective view of the patient monitor in a system with separate devices in accordance with an exemplary embodiment of the present invention;

FIG. 3 is a representation of a display including a trend of physiological data with labeled components in accordance with an exemplary embodiment of the present invention;

FIG. 4 is a representation of a display including a trend of physiological data that exhibits a detected pattern in accordance with an exemplary embodiment of the present invention;

FIG. 5 is a front view of a control panel in accordance with an exemplary embodiment of the present invention;

FIG. 6 is a front view of a control panel in accordance with an exemplary embodiment of the present invention;

FIG. 7 is a front view of a control panel in accordance with an exemplary embodiment of the present invention; and

FIG. 8 is a representation of a display wherein portions of a trend are distinguished by different graphic features to designate a position in time in accordance with an exemplary embodiment of the present invention; and

FIG. 9 is a representation of a display wherein detected patterns in a trend are highlighted in accordance with an exemplary embodiment of the present invention.

### DETAILED DESCRIPTION OF SPECIFIC EMBODIMENTS

One or more specific embodiments of the present invention will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Embodiments of the present invention are directed to a user-interface feature for a patient monitoring device. Specifically, present embodiments include a display control feature that facilitates observation and analysis of historical trend data. Specifically, the display control feature automatically finds and displays particular designated events in the historical data so that the events may be analyzed by a user. These events may include alarms, detected patterns (e.g., desaturation patterns), maximum values, minimum values, markers inserted automatically or by users, and so forth. For example, the display control feature may enable a user to automatically scroll, jump, or snap to a particular event by pressing a scroll button, turning a knob, or selecting an icon on a navigable menu. Thus, a user may utilize present embodiments to avoid the inefficiency of methodically scrolling through large amounts (e.g., hours) of trend data (e.g., a continuous chart of SpO₂ values) in search of patterns (e.g., a desaturation patterns) or other events (e.g., alarms). Indeed, in accordance with present embodiments, the user may simply utilize an activation mechanism (e.g., a control knob, button, selectable menu) that coordinates with the display control feature to display events. For example, a control knob may be turned or a button may be pressed to display the last detected desaturation pattern in a trend of SpO₂ data. Further, additional turns of the knob or presses of the button may allow the user to cycle through all or a portion of the detected desaturation patterns and/or other events.

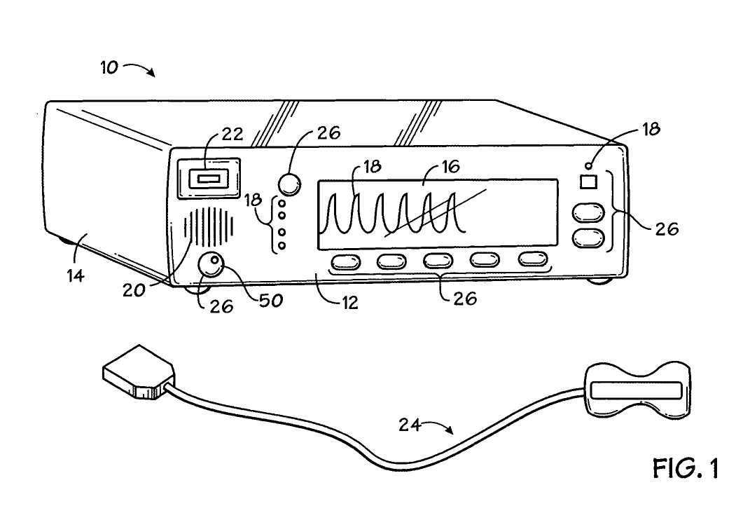

FIG. 1 is a perspective view of a patient monitor 10 in accordance with an exemplary embodiment of the present invention. Specifically, the patient monitor 10 illustrated by FIG. 1 is a pulse oximeter that is configured to detect and monitor blood oxygen saturation levels, pulse rate, and so forth. It should be noted that while the illustrated embodiment includes a pulse oximeter, other embodiments of the present invention may include different types of patient monitors 10. For example, the patient monitor 10 may be representative of a vital signs monitor, a critical care monitor, an obstetrical care monitor, or the like.

The illustrated patient monitor 10 includes a front panel 12 coupled to a body 14 of the monitor 10. The front panel 12 includes a display screen 16 and various indicators 18 (e.g., indicator lights and display screen graphics) that facilitate operation of the monitor 10 and observation of a patient's physiological metrics (e.g., pulse rate). Some of the indicators 18 are specifically provided to facilitate monitoring of a patient's physiological parameters. For example, the indicators 18 may include representations of the most recently measured values for SpO₂, pulse rate, and pulse amplitude. Other indicators 18 may be specifically provided to facilitate operation of the monitor 10. For example, the indicators 18 may include an A/C power indicator, a low battery indicator, an alarm silence indicator, a mode indicator, and so forth. The front panel 12 also includes a speaker 20 for emitting audible indications (e.g., alarms), a sensor port 22 for coupling with a sensor 24 (e.g., a temperature sensor, a pulse oximeter sensor) and other monitor features.

Additionally, the front panel 12 includes various activation mechanisms 26 (e.g., buttons and switches) to facilitate management and operation of the monitor 10. For example, the front panel 12 may include function keys (e.g., keys with varying functions), a power switch, adjustment buttons, an alarm silence button, and so forth. It should be noted that in other embodiments, the indicators 18 and activation mechanisms 26 may be arranged on different parts of the monitor 10. In other words, the indicators 18 and activation mechanisms 26 need not be located on the front panel 12. Indeed, in some embodiments, activation mechanisms 26 are virtual representations in a display or actual components disposed on separate devices.

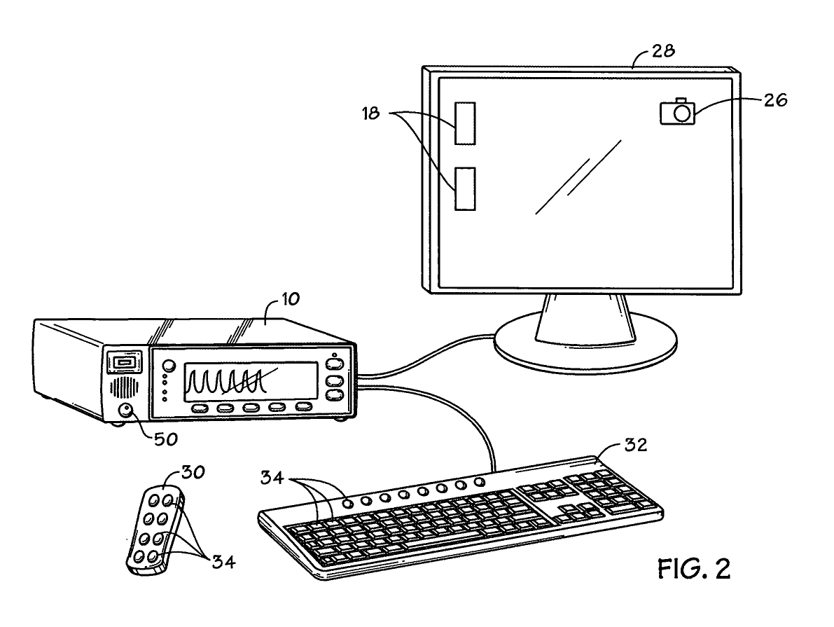

In some embodiments, as illustrated in FIG. 2, the monitor 10 cooperates with separate devices, such as a separate screen 28, a wireless remote 30, and/or a keyboard 32. These separate devices may include some of the indicators 18 and activation mechanisms 26 described above. For example, buttons 34 on the remote 30 and/or keyboard 32 may operate as activation mechanisms 26. Specifically, for example, the buttons 34 may cause the monitor 10 to perform specific operations (e.g., power up, adjust a setting, silence an alarm) when actuated on the separate device. Similarly, the indicators 18 and/or activation mechanisms 26 may not be directly disposed on the monitor 10. For example, the indicators 18 may include icons, indicator lights, or graphics on the separate screen 28 (e.g., a computer screen). Further, the activation mechanisms 26 may include programs or graphic features that can be selected and operated via a display. It should be noted that the separate screen 28 and/or the keyboard 32 may communicate directly or wirelessly with the monitor 10.

As briefly set forth above, embodiments of the present invention include a display control feature that facilitates observation and analysis of historical data. This display control feature may include software or hardware, as well as an activation mechanism to operate the display control feature. For example, FIGS. 1 and 2 include a knob 50 that may be utilized to operate the display control feature. The display control feature may facilitate a user's observation of certain events (e.g., metrics and indications) by eliminating or reducing the time and effort required for a user to find the events by scanning through the data (e.g., trend data). For example, the display control feature may enable a user to turn the knob 50 or to use some other activation mechanism to cause the view provided by the monitor 10 to automatically snap or jump to certain events. In other words, present embodiments may allow a user to snap or jump directly to screens displaying certain events (e.g., alarms, detected patterns, maximum values, minimum values) by activating the display control feature. In one embodiment, a user can turn the knob 50 to scroll through various options and then push the knob 50 to select a particular option (e.g., jump to latest detected desaturation pattern) that causes the display to jump to certain events. In some embodiments, the knob 50 may be replaced by other activation mechanisms. For example, a user may activate the display control feature by pressing a button and/or maneuvering a roller ball. It should be noted that the data to which the monitor 10 snaps or jumps may be displayed by the monitor 10 on the display screen 16 and/or the separate screen 28.

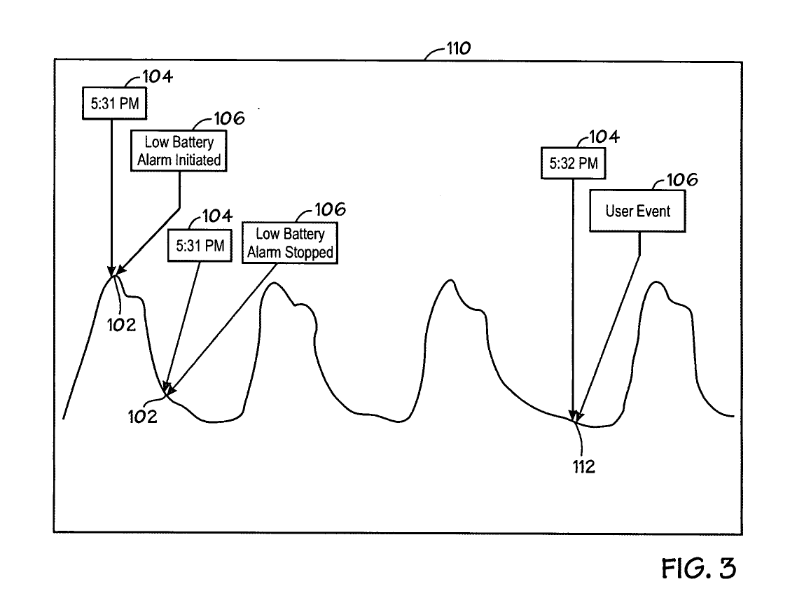

In one embodiment, the monitor 10 may detect and label certain events that can later be readily accessed using the display control feature. Indeed, the events may be continuously detected and labeled by a detection feature of the monitor 10. Additionally, a user may designate certain data points, time periods, and so forth as events. For example, a user may select certain data points for review by highlighting and manually labeling the data. Once such events have been identified, a user may jump or cycle to displays that illustrate the detected events by activating (e.g., depressing, rotating) the activation mechanism (e.g., knob 50) of the display control feature. In a specific example, the monitor 10 may automatically label the moment at which an alarm 102 was initiated by designating the alarm 102 with a timestamp 104 and/or graphic indicator 106, for example, at the corresponding location of the alarm 102 on a trend 108, as illustrated in the exemplary display 110 in FIG. 3. Deactivation of the alarm 102 may also be designated on the trend 108. It should be noted that the alarm 102 may correspond to detected physiological data (e.g., high temperature, low saturation) or any other type of alarm condition (e.g., low battery, sensor off).

In some embodiments, the monitor 10 detects patterns in data (e.g., physiological data) that correspond to certain conditions. For example, present embodiments may detect a cluster of desaturation data or a desaturation pattern that is indicative of ventilation instability or sleep apnea in the patient being monitored. Upon detecting such patterns, the monitor 10 may label (e.g., timestamp, textually indicate, highlight) the initial portion of the pattern and the end portion of the pattern. In other words, the monitor may 10 provide indications of where the pattern begins and where it ends once the pattern is determined to exist. For example, in one embodiment, a pattern portion of a trend may be displayed in reverse video. In another embodiment, the pattern portion of the trend may be displayed with a line having a distinguishing thickness or color. Further, the monitor 10 may essentially diagnose the pattern by labeling it with specific text or other graphical features based on a database of correlations between labels and detected patterns.

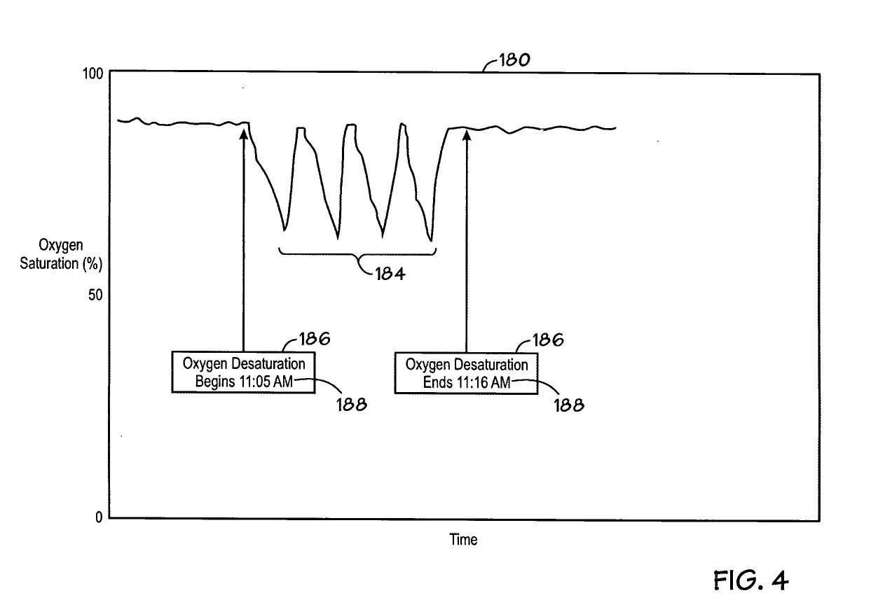

FIG. 4 is a representation of a display 180 that includes a trend of oxygen saturation over time. As illustrated in FIG. 4, the monitor 10 may detect a cluster or pattern 184 of desaturation data, which the monitor 10 may determine is likely indicative of sleep apnea or some other issue. The monitor 10 may then label the pattern 184 with a textual graphic 186 and a timestamp 188 indicating a beginning and end of the detected pattern 184. Such labeling may facilitate rapid diagnosis of a patient by a clinician. For example, the clinician may use present embodiments to simply snap or jump to a display including the pattern 184 (e.g., indication of sleep apnea or ventilation instability) by activating the display control feature (e.g., pressing a button). It should be noted that embodiments of the present invention may utilize systems and methods such as those disclosed in U.S. Pat. No. 6,760,608, U.S. Pat. No. 6,223,064, U.S. Pat. No. 5,398,682, U.S. Pat. No. 5,605,151, U.S. Pat. No. 6,748,252, U.S. application Ser. No. 11/455,408 filed Jun. 19, 2006, U.S. application Ser. No. 11/369,379 filed Mar. 7, 2006, and U.S. application Ser. No. 11/351,787 filed Feb. 10, 2006 to detect certain data patterns. Accordingly, U.S. Pat. No. 6,760,608, U.S. Pat. No. 6,223,064, U.S. Pat. No. 5,398,682, U.S. Pat. No. 5,605,151, U.S. Pat. No. 6,748,252, U.S. application Ser. No. 11/455,408 filed Jun. 19, 2006, U.S. application Ser. No. 11/369,379 filed Mar. 7, 2006, and U.S. application Ser. No. 11/351,787 filed Feb. 10, 2006 are each incorporated herein by reference.

Activation of the display control feature during normal operation of the monitor 10 may cause the monitor 10 to jump or automatically scroll to a display of the most recent detected event. For example, in one embodiment, where no particular event type is designated, a user may press a button or the knob 50 to sequentially jump to all detected events in a set of historical data. Specifically, for example, if no events are detected between the alarm 102 designated in FIG. 3 and when the display control feature is activated, activation of the display control feature may cause the monitor 10 to automatically display historical data of the trend 108 associated with the alarm 102, as illustrated in FIG. 3. However, if events are detected between the time of the alarm and the time of activating the display control feature, the user may use the display control feature to cycle through the events to get to a display of data associated with the alarm 102. For example, a user may create a user designated event 112 by marking a certain portion of data at a point on the trend 108 after the alarm 102 occurred for later review. Such marking may be incorporated as an event by the monitor 10. Accordingly, activation of the display control feature from a current display may cause the monitor 10 to display the user designated event 112 (i.e., the marked data) before proceeding to display the data associated with the alarm 102, which would occur upon additional activation of the display control feature. Indeed, embodiments of the present invention enable a user to cycle through all or a selected subset of events stored by the monitor 10.

A user may select different types of events for the display control feature to cycle through or jump to in accordance with present embodiments. In other words, the display control feature may be configured or programmed by the user such that activation of the display control feature causes the monitor's display to jump to specific types of events and to bypass others. This improves efficiency in viewing and analyzing data by allowing a user to skip over data that is irrelevant or not of interest. For example, a user may only be interested in alarms associated with recognized physiological patterns in the data (e.g., a pattern indicative of sleep apnea). Accordingly, the user may choose to view only labels that include alarms based on recognized physiological patterns and not labels based on equipment alarms (e.g., low battery alarms, sensor disconnected alarms), user markers, or other event types.

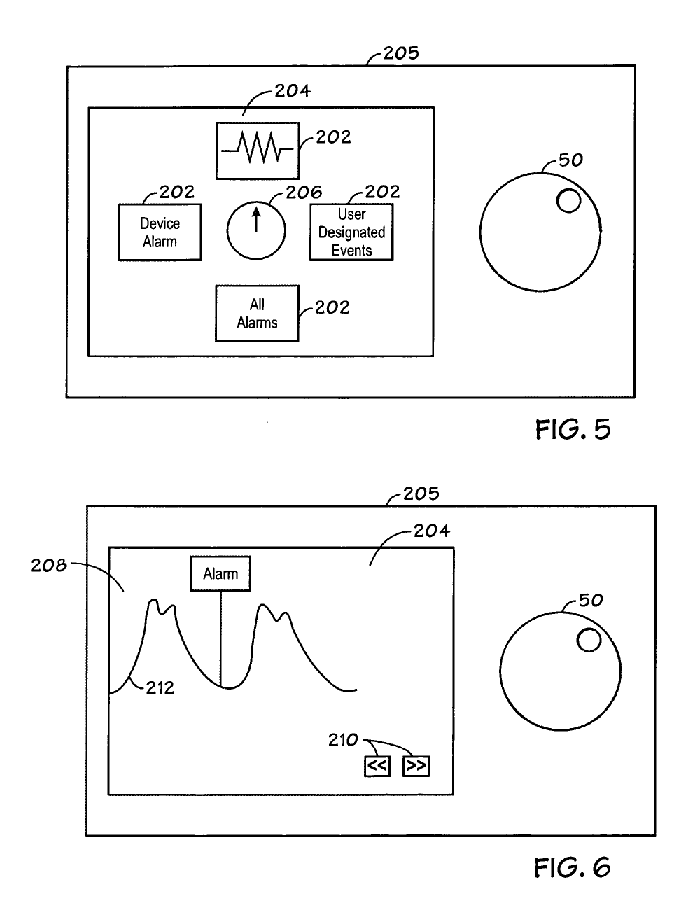

In some embodiments, a user may select particular types of events to snap or jump to when the display control feature is activated. For example, a user may turn the knob 50 to select between various soft menu features 202 that represent different types of events (e.g., events, data pattern types) in a display 204, as illustrated by the front view of a control panel 205 in FIG. 5. Turning the knob 50 may allow the user to navigate a menu or grouping of menu features 202 (e.g., buttons) and select the event type for the display control feature to seek out or jump to when it is activated. For example, a particular event type or set of event types may be selected by pressing the knob 50 when the button or menu item corresponding to the particular event type is highlighted or designated. In a specific example, a user may turn the knob 50 to guide a graphic arrow 206 such that it designates a desired one of the menu features 202, and the user may then depress the knob 50 to select the feature. If the user desires to deselect the feature, the process may be repeated to remove it as a selected feature. Once the event type or types are designated, the knob 50 may be utilized to navigate to a browsing menu 208, as illustrated in FIG. 6, which allows a user to select soft browsing buttons 210 by rotating the knob 50 to highlight the appropriate button and depressing the knob 50. The selection of the soft browsing buttons 210 may activate the display control feature and cause the display to jump to the most recent designated event type in the indicated direction within a trend 212 of historical data.

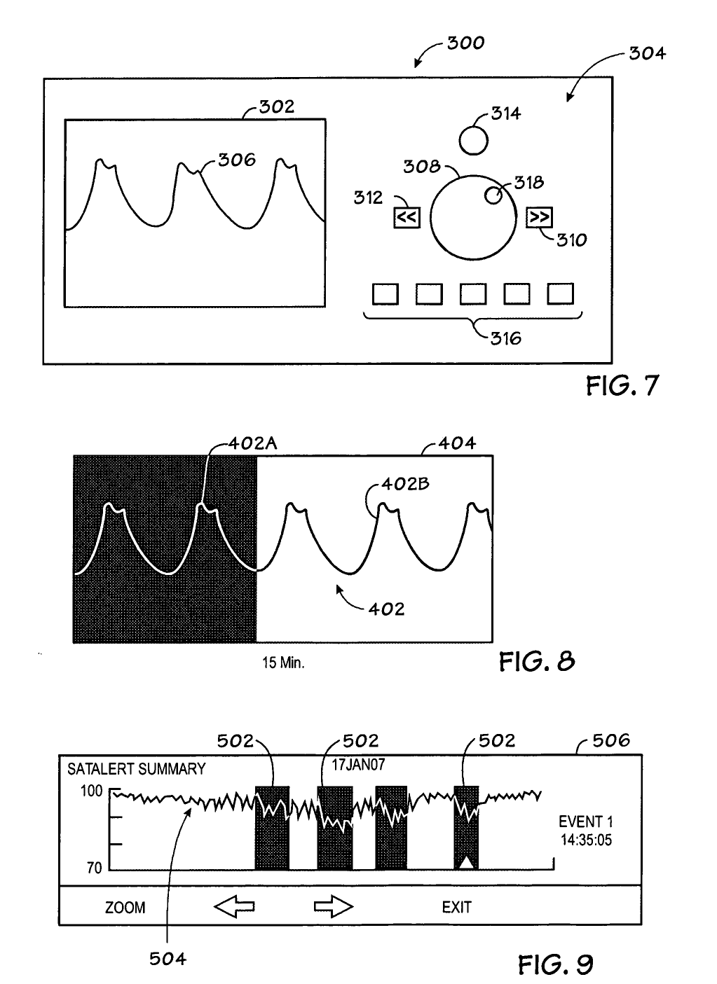

FIG. 7 is a front view of a control panel 300 in accordance with an exemplary embodiment of the present invention. Specifically, the control panel 300 includes a display screen 302 disposed adjacent a plurality of display control mechanisms 304. In the illustrated embodiment, the display screen 302 is displaying a trend 306 of data in an X-Y plot format. In other embodiments, different representations (e.g., bar graph, numerals, text) of the data may be employed. The control mechanisms 304 may include a dial 308, a find-forward button 310, a find-backward button 312, a select button 314, and a plurality of event designator buttons 316. The buttons may be actual buttons or soft buttons. While the illustrated embodiment shows the control mechanisms 304 on the faceplate of an actual monitor, in other embodiments, the control mechanisms 304 may be icons on a display screen and/or features disposed on a remote control that communicates with the actual monitor. In one embodiment, the entire control panel 300 may be a virtual control panel (e.g., a functional graphic) on a display presented on the display screen 302. It should be noted that if the display control feature is configured to only snap or jump to one type of event (e.g., detected desaturation patterns, all detected events), the find-forward 210 and find-backward buttons 212 could be utilized without other features to simplify navigation of the historical data (e.g., trend 306).

The control mechanisms 304 may facilitate navigation through the history of the data (e.g., trend 306) represented on the display screen 302. For example, a user may rotate the dial 308 to slowly scroll through historical data recorded as the trend 306. The display of data may scroll in the direction that the dial 308 is rotated (i.e., counter-clockwise rotation of the dial scrolls the display back in time and clockwise rotation of the dial scrolls the display forward in time). The dial 308 may be substantially flush with the control panel 300, with a circular indentation 318 on the outer perimeter that facilitates rotation by allowing a user to insert a finger tip into the indentation 318 to control movement. In another example, the user may forgo scrolling through historical data by pressing the find-forward button 310 or the find-backward button 312, which may cause the display to jump to a certain event. In one embodiment, the view changes to include the most recent recognized event or selected event type in the direction indicated by the selected control mechanism 304 (e.g., find-backward button 312). For example, the monitor 10 may cause the screen 302 to display the last detected alarm when the find-backward button 312 is depressed or toggled from a real-time or standard operational display of the trending data 306. In another example, pressing the find-forward button 310 from a location in the historical data may cause the display to jump to the next recognized event or selected event type toward the present. If no events are identified between the location being observed and a real-time display, the display may simply jump to the real-time display.

The display control feature may be configured for selective viewing of labels using the event designator buttons 316 or similar input features. For example, a user may select one or more event designator buttons 316 that are associated with particular events of interest (e.g., alarms, alarm types, detected patterns, pattern types, user marks). In a specific example, a user may want the display control feature to operate such that when activated it cycles through sleep apnea patterns detected in a trend of physiological data. Accordingly, the user may select the event designator button 316 corresponding to detected sleep apnea patterns, thus causing the monitor 10 to jump directly to the display of these detected events when the display control feature is activated. In other examples, multiple event types may be selected for such observation. For example, multiple event designator buttons 316 may be activated such that the display control feature snaps to various alarm types and pattern types. Controlling the types of events that the monitor 10 automatically displays upon activation of the display control feature allows for efficient use of the monitor 10.

Embodiments of the present invention may facilitate user observation and analysis of data by establishing a distinction between data of interest (e.g., data having certain notable characteristics, recent data) and other data (e.g., standard data, old data). For example, present embodiments may include graphical features that make a clear distinction between data detected within a designated time period (e.g., within 15 minutes) from a present time and data that is older (e.g., 15 minutes old or older). This may be beneficial in preventing a user (e.g., a clinician) from improperly diagnosing a current situation based on past data. Further, in another example, data of concern (e.g., data exhibiting a pattern of desaturation) may be distinguished from other data. The graphical features may include timestamps 104, graphic indicators 106, color changes in graphic features, blinking text, and so forth. For example, as illustrated in FIG. 8, portions of a trend 402 in a trend display 404 that represent data acquired over fifteen minutes before a present time or old data 402A may be displayed as inverted, while current data 402B or data acquired within fifteen minutes from the present time may be displayed as normal. In another example, as illustrated in FIG. 9, detected patterns 502 in a trend 504 may be highlighted on a trend display 506 to distinguish the patterns 502 from other trend data. In other embodiments, the trend may be displayed in different colors or having varying line thicknesses depending on the nature (e.g., age, pattern) of the associated portions of trend data. Accordingly, when a user snaps back or forward to an event, the user may readily discern the time period in which the event was recorded by observing the indicative graphical feature.

While the invention may be susceptible to various modifications and alternative forms, specific embodiments have been shown by way of example in the drawings and will be described in detail herein. However, it should be understood that the invention is not intended to be limited to the particular forms disclosed. Rather, the invention is to cover all modifications, equivalents and alternatives falling within the spirit and scope of the invention as defined by the following appended claims.

What is claimed is:

1. A monitoring system, comprising:
a monitor configured to receive input relating to patient physiological parameters and to store historical data related to the parameters;
a screen configured to display the historical data corresponding to the patient physiological parameters; and
a display control feature configured to automatically find and display an event in the historical data on the screen when the display control feature is activated.

2. The system of claim 1, wherein the display control feature is configured to recognize and label a pattern in the historical data as the event.

3. The system of claim 2, wherein the pattern includes a desaturation pattern in pulse oximetry data.

4. The system of claim 1, wherein the historical data comprises a trend of SpO₂ values.

5. The system of claim 1, comprising a knob configured to scroll through the historical data.

6. The system of claim 1, comprising an activation mechanism configured to activate the display control feature.

7. The system of claim 6, wherein the activation mechanism is a knob configured to activate the display control feature when pressed.

8. The system of claim 1, wherein the display control feature is configured to display a respective one of a plurality of successive events each time the display control feature is activated.

9. The system of claim 1, wherein the display control feature is configured to graphically distinguish between data from a first time period and data from a second time period.

10. A method, comprising:
receiving input relating to patient physiological parameters;
storing historical data related to the input;
detecting and labeling events in the historical data;
displaying visual data corresponding to the historical data in a normal view;
jumping to a historical view when a display control feature is activated, the historical view including visual data corresponding to the event.

11. The method of claim 10, comprising detecting the event in the historical data based on an alarm.

12. The method of claim 10, comprising detecting the event in the historical data based on a pattern in the data.

13. The method of claim 12, wherein the pattern includes a desaturation pattern in pulse oximetry data.

14. The method of claim 10, comprising initiating the display control feature when input is received from an activation mechanism.

15. The method of claim 10, comprising jumping to a display of a respective one of a plurality of successive events each time the display control feature is activated.

16. The method of claim 10, comprising graphically distinguishing between historical data from a first time period and historical data from a second time period.

17. A method, comprising:
receiving physiological data from a sensor;
identifying a plurality of events in the physiological data; and
jumping to a display of data relating to at least one of the plurality of events when a display control feature is activated.

18. The method of claim 17, comprising labeling each of the plurality of events when the events are identified.

19. The method of claim 18, wherein labeling comprises providing a graphic indicative of event type.

20. The method of claim 18, wherein labeling comprises providing a timestamp for each of the plurality of events.

21. The method of claim 17, comprising detecting a first event of the plurality of events based on a pattern in the physiological data.

22. The method of claim 21, wherein the pattern includes a desaturation pattern in pulse oximetry data.
